(19) United States
(12) Patent Application Publication
(10) Pub. No.: US 2017/0311806 A1
(43) Pub. Date: Nov. 2, 2017

(54) SELF ALIGNING FIBER OPTIC BEAM SHAPING SYSTEM

(71) Applicant: Corning Incorporated, Corning, NY (US)

(72) Inventors: Lovell Elgin Comstock, II, Charlestown, NH (US); William Spencer Klubben, III, Corning, NY (US); Daniel Max Staloff, Rochester, NY (US)

(21) Appl. No.: 15/488,982

(22) Filed: Apr. 17, 2017

Related U.S. Application Data
(60) Provisional application No. 62/329,448, filed on Apr. 29, 2016.

(51) Int. Cl. A61B 5/00; G02B 6/26; F21V 8/00; A61B 1/07; H04N 7/18; G06K 9/62

(52) U.S. Cl. CPC A61B 5/0084; G02B 6/0008; A61B 5/0066; A61B 1/07; G02B 6/262; A61B 2562/0233; A61B 2562/146; G06K 9/6202; H04N 7/183

(57) ABSTRACT

A beam-shaping optical system includes a sheath defining a central cavity having an inner wall, an optical fiber positioned within the cavity and engaged with the inner wall of the sheath, and a beam-shaping insert positioned within the sheath and engaged with the inner wall of the sheath. The beam-shaping insert includes a beam-shaping element with a reflective element aligned with an optical axis of the optical fiber. The optical fiber is configured to emit an electromagnetic beam toward the beam-shaping element and the beam-shaping element is configured to reflect the electromagnetic beam externally to the beam-shaping insert.

This application claims the benefit of priority under 35 U.S.C. §119 of U.S. Provisional Application Ser. No. 62/329,448 filed on Apr. 29, 2016, the content of which is relied upon and incorporated herein by reference in its entirety.

### BACKGROUND

The present disclosure relates to optical probes and in particular to a self-aligning beam-shaping system for use in an optical coherence tomography probe.

Optical coherence tomography (OCT) is used to capture a high-resolution cross-sectional image of biological tissues and is based on fiber-optic interferometry. The core of an OCT system generally is a Michelson interferometer, which typically includes a first optical fiber which is used as a reference arm and a second optical fiber which is used as a sample arm. The sample arm includes the sample to be analyzed, as well as a probe that contains optical components therein. A light source upstream of the probe provides light used in imaging. A photodetector is arranged in the optical path downstream of the sample and reference arms. The probe is used to direct light into or onto the sample and then to collect scattered light from the sample.

Because the probe typically needs to be inserted into a small cavity of the body, it preferably is small. However, conventional alignment methods employ ferrules or other alignment aids, thus increasing the overall diameter of the optical probe.

### SUMMARY

According to one embodiment of the present disclosure, an optical probe includes a sheath having an inner wall defining a central cavity, an optical fiber positioned within the cavity and engaged with the inner wall, and a beam-shaping insert positioned within the cavity and engaged with the inner wall. The beam-shaping insert includes at least one beam-shaping element with a reflective element aligned with an optical axis of the optical fiber. An electromagnetic beam emitted from the optical fiber is reflected by the reflective element of the beam-shaping element.

According to another embodiment of the present disclosure, an optical coherence tomography probe includes a sheath having an inner wall defining a central cavity, an optical fiber positioned within the central cavity and in direct contact with the inner wall of the sheath, and a beam-shaping insert positioned within the cavity and engaged with the inner wall. The beam-shaping insert includes at least one beam-shaping element having a reflective element aligned with an optical axis of the optical fiber. An electromagnetic beam emitted from the optical fiber is reflected by the reflective element.

According to yet another embodiment of the present disclosure, a method of forming an optical probe includes positioning an optical fiber substantially concentrically through a proximal aperture of a sheath such that the optical fiber and an inner wall of the sheath are engaged, positioning a beam-shaping insert substantially concentrically through a distal aperture of the sheath such that the beam-shaping insert and the inner wall are engaged, adjusting a distance and an orientation between the optical fiber and the beam-shaping insert to align an output face of the optical fiber with the beam-shaping insert along an optical axis of the optical probe, and securing the optical fiber and the beam-shaping insert to the sheath. The optical axis extends through the proximal aperture and the distal aperture of the sheath.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

FIG. 1A is an elevated exploded view of an optical probe according to various embodiments described herein;

FIG. 1B is an elevated cross sectional view of the optical probe depicted in FIG. 1A in assembly taken at line IB of FIG. 1A according to various embodiments described herein;

FIG. 1C is an elevated cross sectional view of the optical probe depicted in FIG. 1A in assembly taken at line IB of FIG. 1A according to various embodiments described herein;

FIG. 2 is an enlarged cross sectional view of the optical probe taken at line IB of FIG. 1A according to various embodiments described herein;

FIG. 3 is a schematic diagram of an OCT alignment system that includes the optical probe according to various embodiments described herein; and

FIG. 4 is a schematic diagram of an OCT system that includes an optical probe according to various embodiments described herein.

### DETAILED DESCRIPTION

Reference will now be made in detail to the present preferred embodiments, examples of which are illustrated in the accompanying drawings. Whenever possible, the same reference numerals will be used throughout the drawings to refer to the same or like parts.

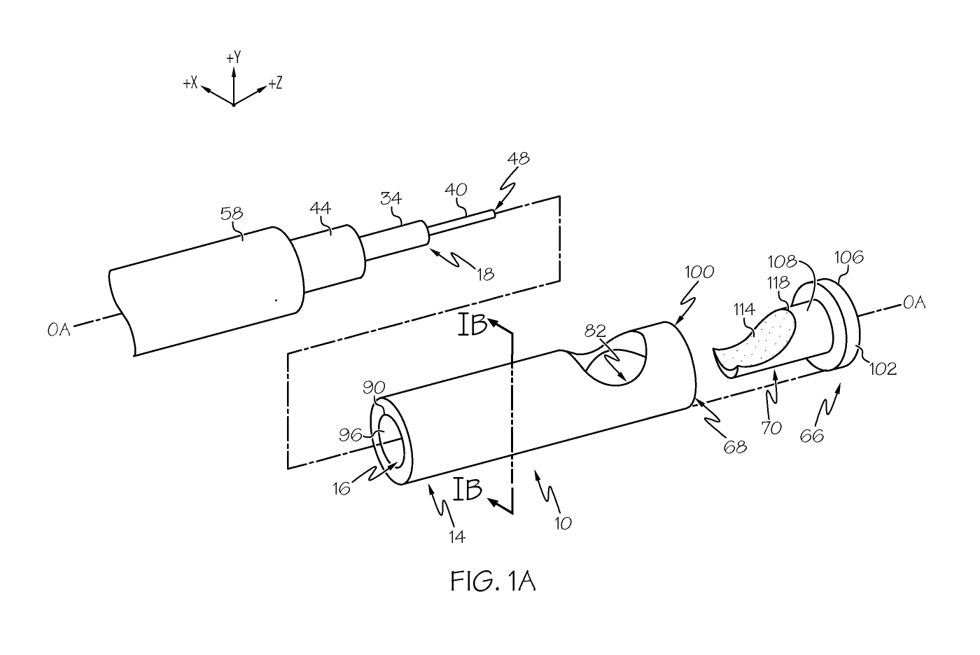

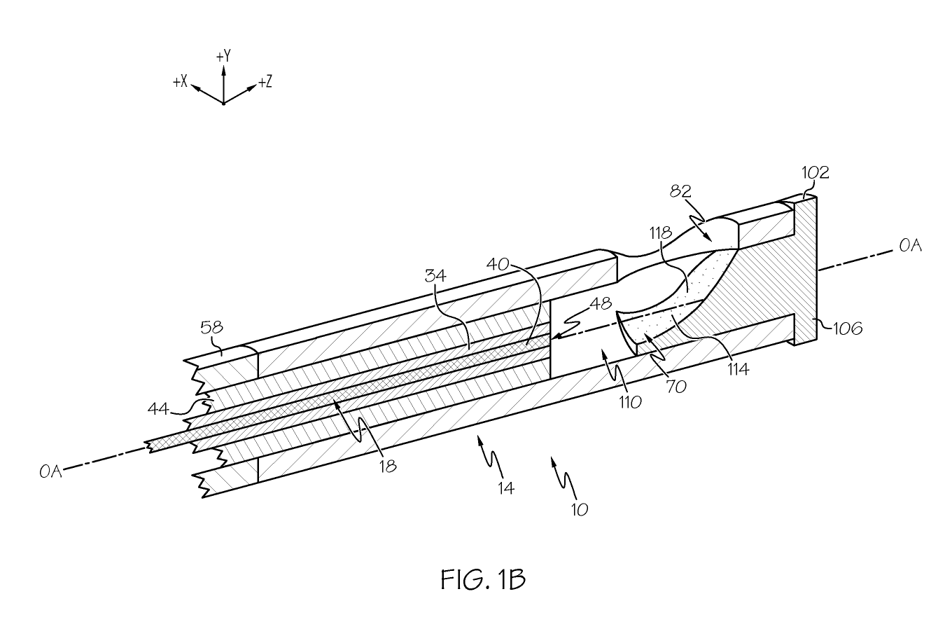

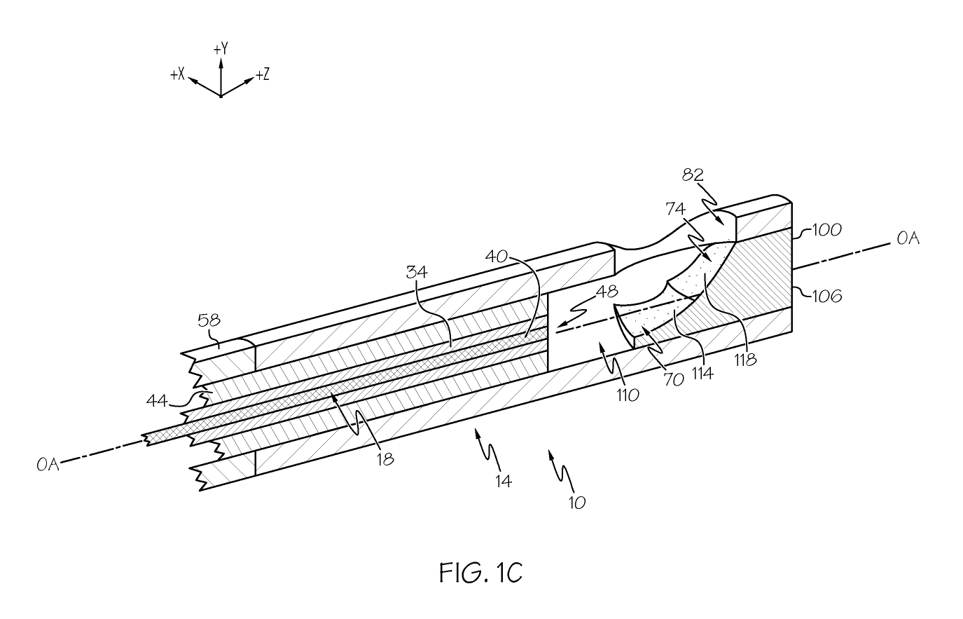

For purposes of description herein, the terms “upper,” “lower,” “right,” “left,” “rear,” “front,” “vertical,” “horizontal,” and derivatives thereof shall relate to an optical probe 10 as oriented in FIG. 1A, unless stated otherwise. However, it is to be understood that the optical probe 10 may assume various alternative orientations, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Depicted in FIGS. 1A-4 is an embodiment of the beam-shaping optical probe 10. The optical probe may be suitable for use in OCT and the making of OCT images, for example. The optical probe 10 includes a sheath 14 defining a central cavity 16 within which an optical fiber 18 is disposed. The optical fiber 18 has a central axis 30 around which a cladding 34, a core 40, and a coating 44 are positioned. In various embodiments the coating 44 is polymeric, but may also comprise metal. The optical fiber 18 includes a fiber end 48 configured to emit an electromagnetic beam 52. The electromagnetic beam 52 may be a light beam (e.g., visible, ultraviolet, infrared or light). The electromagnetic beam 52 is emitted along an optical axis OA defined by the optical probe 10. In assembly, the optical fiber 18 enters the optical probe 10 through a torsional maintaining element 58. A beam-shaping insert 66 is positioned at a distal end 68 of the optical probe 10 and defines a beam-shaping element 70.

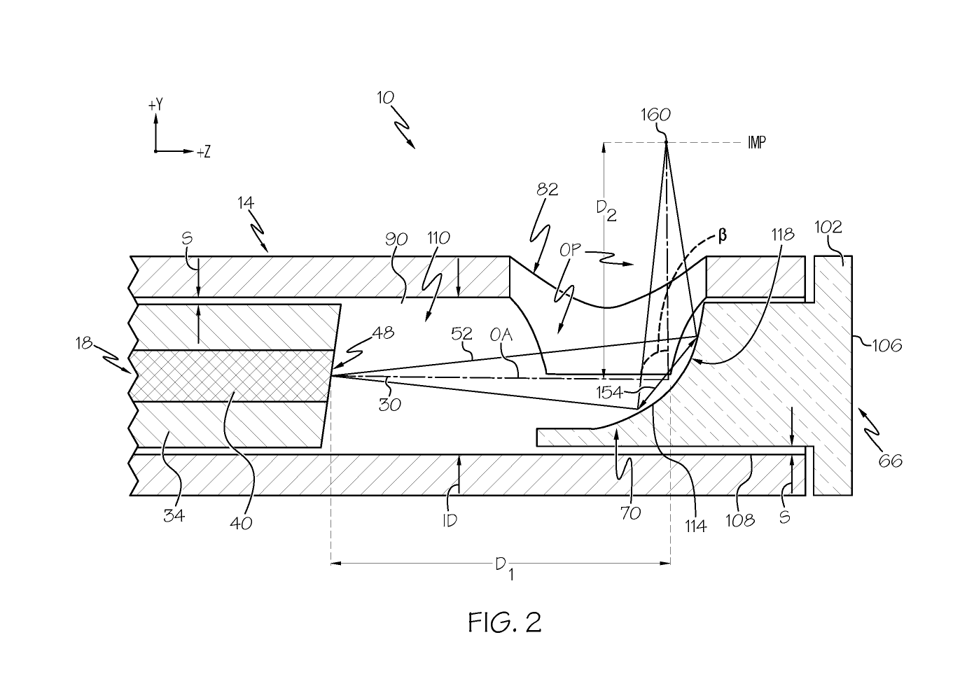

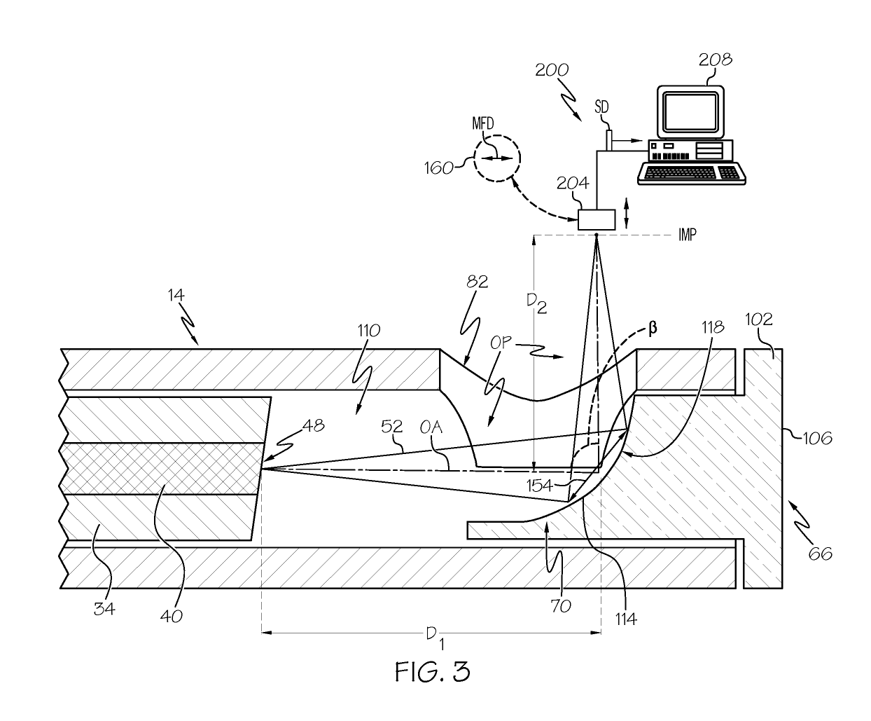

Referring now to FIGS. 1A-1C and 2, the sheath 14 sheath includes an aperture 82, sometimes referred to as a window, through which the electromagnetic beam 52 (FIG. 3) may exit and enter the optical probe 10. Optionally, the aperture 82 may include a transparent material through which the electromagnetic beam 52 can pass, yet prevents foreign matter from entering the optical probe 10. The central cavity 16 of the sheath 14 is defined by an inner wall 90 and has an inner diameter ID. In various embodiments, the inner diameter ID is less than about 300 μm. For example, the inner diameter ID may be between about 125 μm and about 300 μm. In some particular embodiments, the inner diameter ID is between about 130 μm and about 160 μm. A first end, or proximal end, of the sheath 14 defines a proximal aperture 96 and a second end, or distal end 68, of the sheath 14 defines a distal aperture 100. Although depicted in FIGS. 1A-1C and 2 as being made of a single tubular structure, it should be understood that in some embodiments, the sheath 14 is made up of a two portions.

The sheath 14 may be made of a transparent or opaque material. In some embodiments, the sheath 14 may be made of a polymeric material such as latex, polyethylene, or polyurethane or a metal such as 304 or 306 stainless steel. In still other embodiments, the sheath 14 may be made of glass.

In assembly, the optical fiber 18 travels through the torsional maintaining element 58 from an upstream light source (not shown) into the sheath 14. Once the optical fiber 18 enters the sheath 14 through the torsional maintaining element 58, it is positioned within the central cavity 16 of the sheath 14. The outer optical fiber surface is configured to engage and precisely mirror the inner wall 90 of the sheath 14 such that the optical fiber 18 fits within the central cavity 16 in a substantially concentric manner. In various embodiments, the optical fiber 18 is in direct contact with the inner wall 90 of the sheath 14. For example, the coating 44 of the optical fiber 18 may directly contact the inner wall 90 of the sheath 14. In some embodiments, however, the optical fiber 18 is void of a coating on the portion engaged with the inner wall 90 of the sheath 14, and the cladding 34 may directly contact the inner wall 90 of the sheath 14. In still other embodiments, the optical fiber 18 engages with the inner wall 90 through an adhesive interface.

Because in various embodiments the optical fiber 18 engages with the inner wall 90 of the sheath 14, there is no need for a ferrule or other alignment aid, which enables the overall diameter of the optical probe 10 to be reduced. For instance, an outer diameter of the optical probe 10 may be less than about 750 μm, less than about 600 μm, less than about 500 μm, less than about 400 μm, or even less than about 300 μm. In particular embodiments, the outer diameter of the optical probe 10 is between about 130 μm and about 500 μm, between about 200 μm and about 450 μm, or between about 300 μm and about 400 μm.

The beam-shaping insert 66 is configured to be inserted into the central cavity 16 of the distal end 68 of the sheath 14 through the distal aperture 100. During insertion of the beam-shaping insert 66, a flange 102 may be placed in abutting contact with the sheath 14 and a beam-shaping surface 108 is in contact with the inner wall 90. It will be understood that various embodiments of the optical probe 10 and beam-shaping insert 66, such as the embodiment depicted in FIG. 1C, do not include a flange 102. The flange 102 is positioned on the beam-shaping insert 66 such that during insertion, the flange 102 contacts the sheath 14 as the beam-shaping element 70 is positioned proximate the aperture 82. In this manner, the flange 102 may aid in the positioning of the beam-shaping insert 66 within the sheath 14 as well as the beam-shaping element 70. Optionally, a forward surface 106 of the beam-shaping insert 66 and/or the flange 102 includes one or more markings (e.g., degree dial, an index line, hash marks) designed to aid an operator in correctly orienting the beam-shaping insert 66 within the sheath 14. Additionally or alternatively, the sheath 14 may include the same, similar, or complimentary markings as the forward surface 106 to aid in orientation of the beam-shaping insert 66. Orientation of the beam-shaping insert 66 within the sheath 14 is performed such that the beam-shaping element 70 is aligned with the optical axis OA of the optical probe 10 and the aperture 82 of the sheath 14. A gap 110 is defined between the fiber end 48 and the beam-shaping insert 66 when in assembly. The gap 110 may comprise only air, but also optically transmissive liquids and solids which may aid in the shaping of the electromagnetic beam 52. For example, a gas, an adhesive, or saline may be positioned within and fill the gap 110 within the central cavity 16 between the optical fiber 18 and the beam-shaping insert 66.

In various embodiments, the beam-shaping insert 66 includes a polymeric composition. Exemplary polymeric materials for the beam-shaping insert 66 include ZEONOR® (available from Zeon Chemicals L.P., Louisville, Ky.), polyetherimide (PEI), polyethylene, polypropylene, polycarbonate, engineered polymers (e.g., liquid crystal), as well as any other polymeric material or combination of polymeric materials capable of forming the beam-shaping insert 66 and producing a smooth surface. In other embodiments, the beam-shaping insert 66 may include metals, ceramics, glasses, or composites thereof. The beam-shaping insert 66 is capable of formation by conventional manufacturing techniques such as injection molding, casting, machining, thermoforming, or extrusion.

The optical fiber 18 and the beam-shaping insert 66 are each configured to engage the central cavity 16 of the sheath 14. The diameters of the optical fiber 18 and the beam-shaping insert 66 are substantially similar (e.g., less than about 10 micron difference) to that of the inner diameter ID of the sheath 14 such that the optical fiber 18 and the beam-shaping insert 66 engage the sheath 14 in a substantially concentric manner. In assembly, a spacing S is defined between the optical fiber 18 and the inner wall 90 or the beam-shaping surface 108 and the inner wall 90. The spacing S may be between about 0.1 microns and about 40 microns, or between about 1 micron and about 20 microns. In specific examples, the spacing S may be less than about 10 microns, less than about 9 microns, less than about 8 microns, less than about 7 microns, less than about 6 microns, less than about 5 microns, less than about 4 microns, less than about 3 microns, less than about 2 microns, and less than about 1 micron. The tight tolerance of the spacing S between the inner wall 90 and the beam-shaping surface 108 and the optical fiber 18 ensures that the positional accuracy in the X- and Y-axes of the beam-shaping insert 66 and the optical fiber 18 is nearly perfect upon insert into the sheath 14. By forming the optical fiber 18 and beam-shaping insert 66 as self-aligning components within the sheath 14, manufacturing time and effort related to positioning and aligning of the optical probe 10 may be decreased. Additionally, by positioning the optical fiber 18 within the aperture, along which the electromagnetic beam 52 is emitted, may be quickly aligned to the optical axis OA of the optical probe 10 due to the high concentricity between the optical fiber 18 and the inner wall 90 of the probe 10 without the use of a ferrule or other alignment aid. The optical fiber 18 and the sheath 14 are configured to align such that an offset between the central axis 30 of the optical fiber 18 and the optical axis OA of the optical probe 10 is less than about 7 microns, about 6 microns, about 5 microns, about 4 microns, about 3 microns, about 2 microns, about 1 micron, and less than about 0.1 microns. It will be understood that although the depicted embodiment of the sheath 14 and the beam-shaping insert 66 are each substantially cylindrical in shape, the sheath 14 and the beam-shaping insert 66 may take a variety of shapes configured to precisely mate (e.g., cuboid, rectangular, or triangular).

Still referring to FIGS. 1A-1C and 2, the beam-shaping element 70 is integrally defined by the beam-shaping insert 66 such that in assembly, the beam-shaping element 70 is positioned inside of the central cavity 16 of the sheath 14. The beam-shaping element 70 includes a reflective element 114 positioned on a curved surface 118 defined from the beam-shaping insert 66. The beam-shaping insert 66 extends in an upwardly and inwardly curved manner with respect to the forward surface 106 to define the curved surface 118. In various embodiments, the beam-shaping element 70 may be spherical or aspherical in shape. Example aspherical surfaces include bi-conic, parabolic, hyperbolic, etc. In particular embodiments, the beam-shaping element 70 may be a spherical, aspherical, Zernike, rotationally non-symetric or non-uniform rational Basis spline (NURB), or conical element. Zernike elements and NURB elements include a curved surface 118 that may be represented by a Zernike polynomial or NURB model, respectively.

In various embodiments, aspheric surfaces have bilateral symmetry in both X and Y directions, may not have rotational symmetry. In some embodiments, the surface form that does not include rotational symmetry may be referred to as a bi-conic surface. The bi-conic surface may be represented according to the equation:

$$z = \frac{( CUX ) {x}^{2} + ( CUY ) {y}^{2}}{1 + \sqrt{1 - ( 1 + KX ) {( CUX )}^{2} {x}^{2} - ( 1 + KY ) {( CUY )}^{2} {y}^{2}}} + AR {\{ ( 1 - AP ) {x}^{2} + ( 1 + AP ) {y}^{2} \}}^{2} + BR {\{ ( 1 - BP ) {x}^{2} + ( 1 + BP ) {y}^{2} \}}^{3} + CR {\{ ( 1 - CP ) {x}^{2} + ( 1 + CP ) {y}^{2} \}}^{4} + DR {\{ ( 1 - DP ) {x}^{2} + ( 1 + DP ) {y}^{2} \}}^{5}$$

where z is the sag of the surface parallel to the z-axis; CUX is the curvature in x; CUY is the curvature in Y; KX is the conic coefficient in x; KY is the conic coefficient in y; AR, BR, CR, and DR are the rotationally symmetric portion of the 4^{th}, 6^{th}, 8^{th}, and 10^{th} order deformation from the conic; and AP, BP, CP, and DP represent the non-rotationally symmetric components of the 4^{th}, 6^{th}, 8^{th}, and 10^{th} order deformation from the conic.

In various embodiments, the beam-shaping element 70 is substantially conic in shape and curves inwardly toward the optical axis OA of the optical probe 10. The conic shape of the beam-shaping element 70 is defined by a radius of curvature and conic constant along an axis of the beam-shaping element 70 with respect to the optical axis OA of the optical probe 10.

In order to properly shape the electromagnetic beam 52, the beam-shaping element 70 may have a radius of curvature along the X-axis that is the same or different (e.g., bi-conic) than a radius of curvature in the Y-axis. The radius of curvature of the X- and Y-axes of the curved surface 118 of the beam-shaping element 70 may have an absolute value of between about 0.5 millimeters and about 10 millimeters, and more specifically, about 1.0 millimeter to about 4.0 millimeters. The conic constant of the X- and Y-axes of the beam-shaping element 70 may independently range from about 1 to about −2, and more specifically between about 0 and about −1. It should be understood that the radii and conic constants of the curved surface 118 explained above describe the overall shape of the beam-shaping element 70, and do not necessarily reflect local radii or conic constants of the curved surface 118. The radius of curvature of the X-axis and Y-axis of the beam-shaping element 70 may be adjusted independently in order to correct for any material disposed around the optical probe 10. The conic shape of the beam-shaping element 70 may be decentered along the Y- or Z-axes between about 0.01 millimeters and about 0.8 millimeters. Additionally, the conic shape of the beam-shaping element 70 may have a rotation between the Y- and Z-axes of between about 70° and 120°.

The beam-shaping element 70 is configured to collect and shape (e.g., collimate, converge, focus, and/or change the optical path of) through reflection the electromagnetic beam 52 (FIG. 3) emitted from the optical fiber 18, as explained in greater detail below. Positioned on the curved surface 118 of the beam-shaping element 70 is the reflective element 114. The reflective element 114 may be a dielectric coating, a metal coating, or an enhanced metal coating. Exemplary metal coatings include silver, gold, aluminum, platinum and other lustrous metals capable of reflecting the beam 52. Dielectric coatings may include one or more dielectric stack having alternating layers of SiO₂ and at least one of Ta₂O₅, NbO₅, TiO₂, and HfO₂. Further, enhanced metal coatings may include a combination of one or more of the previously described metals and/or dielectrics. For example, the reflective element 114 may include a base layer of silver with one or more dielectric stacks positioned thereon. The reflective element 114 may also include a capping layer to protect it from environmental conditions (e.g., water, oxygen, and/or sterilization procedures). Additionally or alternatively, the reflective element 114 may include a barrier layer. The barrier layer may serve to both adhere the reflective element 114 to the curved surface 118 of the beam-shaping insert 66 as well as protect the beam-shaping insert 66 from damage in high power embodiments of the electromagnetic beam 52. The barrier layer may comprise layers of chromium, aluminum, and alumina, each layer having a thickness of between about 10 nm and about 50 nm. The reflective element 114 is positioned on the beam-shaping element 70 such that the emitted beam 52 is reflected externally to the beam-shaping insert 66, and not within it.

Referring now to the depicted embodiment of FIG. 1C, the beam-shaping insert 66 may not define the flange 102, but rather be configured to slide completely into the sheath 14 through the distal aperture 100. In such an embodiment, the beam-shaping element 70 may be quickly and accurately positioned by making the forward surface 106 of the beam-shaping insert 66 flush with the distal end 68 of the sheath 14. As explained above, the forward surface 106 may include markings to aid in orienting the beam-shaping insert 66 inside the sheath 14.

Also shown in FIG. 1C, the beam-shaping insert 66 may define a second beam-shaping element 74 in addition to the beam-shaping element 70. The second beam-shaping element 74 is depicted as being defined above the beam-shaping element 70, but may be defined to a side, below, or within the beam-shaping element 70. The second beam-shaping element 74 may be substantially similar to the beam-shaping element 70 (i.e., the reflective element 114 positioned on the curved surface 118), or may include a different reflection system. The second beam-shaping element 74 may have a different radius of curvature and/or conic constant along the X- and/or Y-axes than the beam-shaping element 70. In various embodiments, the second beam-shaping element 74 is configured to shape the electromagnetic beam 52 differently (e.g., in a different direction or to a different working distance) than the beam-shaping element 70.

Referring now to FIG. 2, in operation, the optical fiber 18 is configured to act as a wave guide for electromagnetic radiation, specifically light at an operating wavelength λ. The optical fiber 18 carries light from an upstream light source (not shown) to the fiber end 48 where the light is emitted as the electromagnetic beam 52. In one embodiment, the operating wavelength λ includes an infrared wavelength such as one in the range from about 830 nanometers to about 1,600 nanometers, with exemplary operating wavelengths λ being about 1300 nanometers and about 1560 nanometers. In various embodiments, the operating wavelengths λ may be as low as about 700 nanometers. The optical fiber 18 may be a single mode or a multimode configuration. The optical fiber 18 may have a mode field diameter of between about 9.2 microns+/−0.4 microns at a wavelength of 1310 nanometers and have a mode field diameter of about 10.4 microns+/−0.5 microns at 1550 nanometers. The diameter of the cladding 34 may be between about 120 microns and about 130 microns.

The optical fiber 18 is configured to couple with the inner wall 90 of the sheath 14 such that when the optical fiber 18 is within the proximal aperture 96, the electromagnetic beam 52 is emitted from the fiber end 48 on an optical path OP that is both substantially coaxial with the optical axis OA of the optical probe 10, and directed toward the beam-shaping element 70. As the electromagnetic beam 52 is emitted from the fiber end 48, it propagates through the gap 110 and the diameter of the optical path OP widens with increasing distance from the fiber end 48. A distance D₁ between the fiber end 48 and the reflective element 114 of the beam-shaping element 70 is set based on a desired size of a beam spot 154. The beam spot 154 is the area of light the electromagnetic beam 52 forms as it strikes the beam-shaping element 70. The beam spot 154 grows in diameter with increasing distance D₁ from the fiber end 48. In order for the beam-shaping element 70 to properly shape the electromagnetic beam 52, the beam spot 154 must have the proper diameter when contacting the reflective element 114 (e.g., approximately half the diameter of the reflective element 114). Accordingly, the fiber end 48 must be placed a predetermined distance from the beam-shaping element 70 for the electromagnetic beam 52 to be properly shaped. In various embodiments, the distance D₁ between the fiber end 48 and the reflective element 114 may range between about 0.2 millimeters and about 2.6 millimeters. In one embodiment, the distance D₁ is about 1.314 millimeters. The diameter of the beam spot 154 may range from about 200 microns to about 1600 microns and more specifically, between about 400 microns to about 600 microns.

As the electromagnetic beam 52 enters the beam-shaping element 70, its optical path OP is folded by an angle β from reflection off of the reflective element 114. In the depicted embodiment, the angle β is approximately 90°, but in various embodiments can vary greater than or less than about 25°, about 20°, and about 10° on either side of 90°. The radius of curvature and position of the beam-shaping element 70 determine both the angle β that the optical path OP of beam 52 will be folded by, and also a working distance D₂ to an image plane IMP where the beam 52 converges to form an image spot 160. Accordingly, the emitted beam 52 is shaped into the image spot 160 solely by reflection from the beam-shaping element 70.

Still referring to FIG. 2, the fiber end 48 of the optical fiber 18 may terminate at an angle in order to prevent undesired back reflection of light in the fiber 18. Some embodiments, such as OCT, may be particularly sensitive to back reflections of light which have not been scattered off of a sample to be tested (i.e., reflections from the optical probe 10, fiber end 48, or refractive surfaces along the optical path OP). The back reflected light may lead to distortion in the OCT image because of increased noise and artifacts. Terminating the fiber end 48 at an angle minimizes the coupling of the back reflected light back into the optical fiber 18. The fiber end 48 may be prepared at an angle between about −10° to about 10° relative to an axis perpendicular to the optical axis OA (e.g., the Y-axis in FIG. 2), and more particularly between about 0° to 10° or even about 6° to about 9°. Angling of the fiber end 48 may be accomplished, for example, by cleaving the fiber end 48 before insertion into the sheath 14. In some embodiments, the beam-shaping element 70 may be angled with respect to the optical axis OA of the optical probe 10 in order to compensate for the angled fiber end 48. Additionally or alternatively, the fiber end 48 may include an anti-reflection film to reduce the amount of reflected light absorbed by the optical fiber 18. The anti-reflection film may include a single or multilayer dielectric material configured to cancel light reflected back to the optical probe 10.

In various embodiments, the fiber end 48 of the optical fiber 18 may be locally tapered with respect to the rest of the optical fiber 18. Tapering of the fiber end 48 may be accomplished through laser heating, plasma heating, resistance heating, or flame heating a portion of the optical fiber 18, and placing the fiber 18 in tension. The heated portion of the fiber 18 then necks down as it is pulled. The fiber 18 may be pulled until the fiber 18 is separated or the heated portion of the fiber 18 may be cut while in the necked down position. Tapering of the core 40 may have an axial length along the optical fiber 18 of about 1 millimeter to about 5 millimeters, and in a specific example of about 4 millimeters. The tapering of the fiber end 48 should be such that the fiber end 48 does not experience adiabatic loss. Tapering of the optical fiber 18 at the fiber end 48 may locally increase the mode field diameter of the fiber end 48. The mode field diameter at a beam 52 wavelength of 1310 nanometers of the tapered fiber end 48 may range from about 8 microns to about 40 microns and in specific examples be about 9 microns, about 10 microns, about 11 microns, about 12 microns, about 13 microns, about 14 microns, about 15 microns, about 16 microns, about 17 microns, about 18 microns, about 19 microns, or about 20 microns. The mode field diameter of the fiber end 48 may expand about 5%, about 10%, about 100%, about 400%, or about 500%. Tapering of the optical fiber 18 at the fiber end 48 may locally increase the mode field diameter of the fiber end 48. The mode field diameter at a beam 52 wavelength of 1310 nanometers of the tapered fiber end 48 may range from about 5 microns to about 40 microns and in specific examples be about 9 microns, about 10 microns, about 11 microns, about 12 microns, about 13 microns, about 14 microns, about 15 microns, about 16 microns, about 17 microns, about 18 microns, about 19 microns, or about 20 microns. Tapering and angling the fiber end 48 of the optical fiber 18 may decrease the back reflection from about −10 dB to about −350 dB, and in specific examples to below about −80 dB, −90 dB, −100 dB, −110 dB, −120 dB and below about −130 dB depending on the level of tapering. In other embodiments, the core 40 of the fiber end 48 may be locally expanded in addition to being prepared with an angle.

Referring now to FIG. 3, the optical probe 10 is depicted in use within an OCT alignment system 200. As explained above, light traveling within the optical fiber 18 exits the fiber end 48 and is emitted as the electromagnetic beam 52 along the optical axis OA. The optical path OP of the electromagnetic beam 52 diverges as it passes through the gap 110 until it enters the beam-shaping element 70 and reflects from the reflective element 114. The curvature of the beam-shaping element 70 causes the light to converge uniformly to the image spot 160 due to the curved surface 118. In the depicted embodiment, as the electromagnetic beam 52 converges, it passes through the aperture 82 of the sheath 14 and forms the image spot 160 at the image plane IMP. The working distance D₂ is measured between the horizontal portion of the optical axis OA of the probe 10 and the image plane IMP and may be between about 1 millimeter and about 20 millimeters.

The proper orientation of the optical probe 10 during manufacturing is facilitated by the use of the optical fiber 18, the beam-shaping insert 66, and the OCT alignment system 200. In an exemplary method for alignment of the optical fiber 18, a photodetector 204 (e.g., camera or a rotating slit) can be used to capture at least one image of image spot 160 and generate a detector signal SD representative of the captured image. The captured image(s) can be analyzed, e.g., via a computer 208 that is operably connected to photodetector 204. The computer 208 can be used to analyze and display information about the captured image spot(s) 160. In an example, a plurality of image spots 160 are detected and compared to a reference spot (e.g., as obtained via optical modeling based on the design of the optical probe 10) to assess performance. If the detected image spots 160 are incorrect, an operator assembling the optical probe 10 may adjust a distance in the Z direction between the optical fiber 18 and the beam-shaping insert 66, or use the markings on the forward surface 106 of the beam-shaping insert 66, to adjust its orientation relative to the sheath 14. The use of the optical fiber 18 and the beam-shaping insert 66 allow for near precise alignment of the optical probe 10 in at least the X- and Y-axes direction upon initial assembly due to the high concentricity.

The mode field diameter MFD is a measure of the spot size or beam width of light propagating in a single mode fiber or at another location in an optical system. The mode field diameter MFD within an optical fiber is a function of the source wavelength, fiber core radius and fiber refractive index profile. In the depicted embodiment, the optical probe 10 is capable of producing an image spot 160 having a mode field diameter MFD of between about 5 microns to about 100 microns at 1310 nm at a 1/e² threshold at the image plane IMP. An exemplary mode field diameter at the image plane IMP may be about 9.3 microns.

The position of optical fiber 18 can be axially adjusted within the optical probe 10 (e.g., by moving the optical fiber 18 or beam-shaping insert 66) based on making one or more measurements of image spot 160 until an acceptable or optimum image spot is formed. In an example, the one or more measured image spots 160 are compared to a reference image spot or a reference image spot size. The optical fiber 18 and the beam-shaping insert 66 can then be fixed in their respective aligned positions and orientations within the sheath 14 via one or more attachment methods (e.g., set screws, epoxies, adhesives, UV curable adhesives, friction fit, etc.).

In an exemplary embodiment of optical probe 10, the beam-shaping element 70 has an X-axis radius of curvature of about 1.16 millimeters and an X-axis conic constant of about 0.5858 and a Y-axis radius of curvature of about 1.2935 millimeters and a Y-axis conic constant of about 0.8235. Further, the conic shape of the beam-shaping element 70 is decentered along the Y-axis by about −0.1657 millimeters and is tilted about −60.7996° with respect to an axis perpendicular to the optical axis OA. The distance D₁ between the fiber end 48 and reflective element 114 is about 0.6103 millimeters. The working distance D₂ between the IMP and the optical axis OA is about 2 mm. Such an optical probe is capable of forming the image spot 160 at the working distance D₂ of about 54 μm with a mode field diameter MFD of about 9.3 microns at 1310 nm the 1/e² threshold. The optical probe 10 may be immersed in saline which has a refractive index of about 1.33 at 1310 nm.

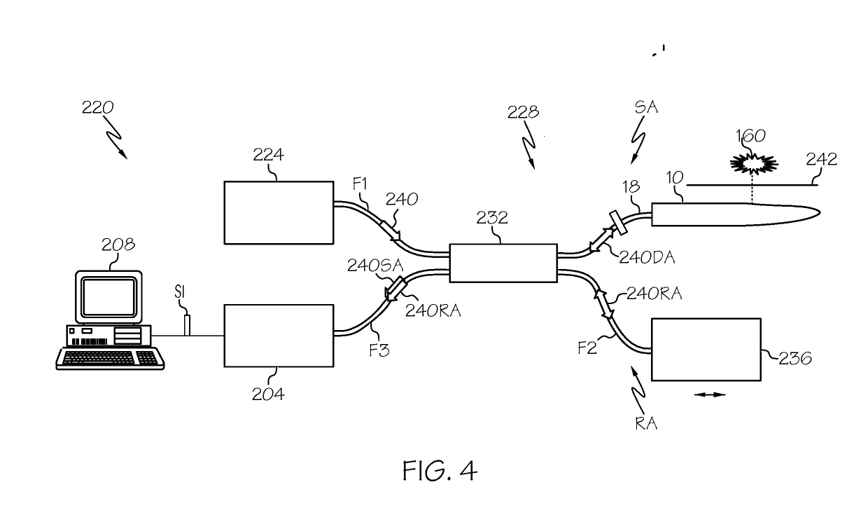

FIG. 4 illustrates an exemplary OCT system 220 that includes an embodiment of the optical probe 10 as disclosed herein. OCT system 220 includes a light source 224 and an interferometer 228. The light source 224 is optically connected to a fiber optic coupler (“coupler”) 232 via a first optical fiber section FI. Optical probe 10 is optically connected to coupler 232 via optical fiber 18 and constitutes the sample arm SA of the interferometer 228. OCT system 220 also includes a movable mirror system 236 optically connected to coupler 232 via an optical fiber section F2. Mirror system 236 and optical fiber section F2 constitute a reference arm RA of the interferometer 228. Mirror system 236 is configured to alter the length of the reference arm, e.g., via a movable mirror (not shown). OCT system 220 further includes the photodetector 204 optically coupled to coupler 232 via a third optical fiber section F3. Photodetector 204 in turn is electrically connected to computer 208.

In operation, light source 224 generates light 240 that travels to interferometer 228 over optical fiber section FI. The light 240 is divided by coupler 232 into light 240RA that travels in reference arm RA and light 240SA that travels in sample arm SA. The light 240RA that travels in reference arm RA is reflected by mirror system 236 and returns to coupler 232, which directs the light to photodetector 204. The light 240SA that travels in sample arm SA is processed by optical probe 10 as described above (where this light was referred to as just emitted beam 52) to form image spot 160 on or in a sample 242. The resulting scattered light is collected by optical probe 10 and directed through optical fiber 18 to coupler 232, which directs it (as light 240SA) to photodetector 204. The reference arm light 240RA and sample arm light 240SA interfere and the interfered light is detected by photodetector 204. Photodetector 204 generates an electrical signal SI in response thereto, which is then sent to computer 208 for processing using standard OCT signal processing techniques.

The optical interference of light 240SA from sample arm SA and light 240RA from reference arm RA is detected by photodetector 204 only when the optical path difference between the two arms is within the coherence length of light 240 from light source 224. Depth information from sample 242 is acquired by axially varying the optical path length of reference arm RA via mirror system 236 and detecting the interference between light from the reference arm and scattered light from the sample arm SA that originates from within the sample 242. A three-dimensional image is obtained by transversely scanning in two dimensions the optical path in the sample arm SA. The axial resolution of the process is determined by the coherence length.

It should be understood that although the use of the optical probe 10 was described in connection with only one OCT technique, the optical probe 10 may be used in a wide variety of applications, including other OCT techniques (e.g., Frequency Domain OCT, Spectral Domain OCT).

While the embodiments disclosed herein have been set forth for the purpose of illustration, the foregoing description should not be deemed to be a limitation on the scope of the disclosure or the appended claims. It will be apparent to those skilled in the art that various modifications and variations can be made without departing from the spirit or scope of the claims.

It will be understood by one having ordinary skill in the art that construction of the described invention and other components is not limited to any specific material. Other exemplary embodiments of the invention disclosed herein may be formed from a wide variety of materials, unless described otherwise herein. In this specification and the amended claims, the singular forms “a,” “an,” and “the” include plural reference unless the context clearly dictates otherwise.

Where a range of values is provided, it is understood that each intervening value, to the tenth of the unit of the lower limit, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It will be apparent to those skilled in the art that various modifications and variations can be made without departing from the spirit or scope of the claims.

What is claimed is:

1. An optical probe, comprising:
a sheath having an inner wall defining a central cavity;
an optical fiber positioned within the cavity and engaged with the inner wall; and
a beam-shaping insert positioned within the cavity and engaged with the inner wall, the beam-shaping insert comprising at least one beam-shaping element with a reflective element aligned with an optical axis of the optical fiber, whereby an electromagnetic beam emitted from the optical fiber is reflected by the reflective element.

2. The optical probe of claim 1, the optical fiber comprising a fiber end, wherein the fiber end of the optical fiber is prepared at an angle between about −10° and about 10° relative to an axis perpendicular to an optical axis of the optical probe.

3. The optical probe of claim 2, wherein the fiber end of the optical fiber is prepared at an angle of about 0° relative to the axis perpendicular to the optical axis of the optical probe.

4. The optical probe of claim 1, the reflective element of the at least one beam-shaping element comprising at least one of a dielectric, metal, and enhanced metal coating.

5. The optical probe of claim 1, the beam-shaping element comprising a bi-conic element.

6. The optical probe of claim 1, the sheath having an inner diameter of less than about 300 μm.

7. The optical probe of claim 1, the sheath having an inner diameter of between about 125 μm and about 300 μm.

8. An optical probe, comprising:
a sheath having an inner wall defining a central cavity;
an optical fiber positioned within the central cavity and in direct contact with the inner wall of the sheath; and
a beam-shaping insert positioned within the cavity and engaged with the inner wall, the beam-shaping insert comprising at least one beam-shaping element having a reflective element aligned with an optical axis of the optical fiber, whereby an electromagnetic beam emitted from the optical fiber is reflected by the reflective element.

9. The optical probe of claim 8, the sheath comprising a proximal aperture and a distal aperture, wherein the optical fiber extends through the proximal aperture.

10. The optical probe of claim 8, the beam-shaping element comprising a spheric, aspheric, Zernike, NURB, or conic element.

11. The optical probe of claim 8, the beam-shaping element comprising a bi-conic element.

12. The optical probe of claim 8, further comprising an optically transparent medium selected from the group consisting of a gas, an adhesive, and saline positioned within the central cavity between the optical fiber and the beam-shaping insert.

13. A method of forming an optical probe, comprising:
positioning an optical fiber substantially concentrically through a proximal aperture of a sheath such that the optical fiber and an inner wall of the sheath are engaged;
positioning a beam-shaping insert substantially concentrically through a distal aperture of the sheath such that the beam-shaping insert and the inner wall are engaged;
adjusting a distance and an orientation between the optical fiber and the beam-shaping insert to align an output face of the optical fiber with the beam-shaping insert along an optical axis of the optical probe, the optical axis extending through the proximal aperture and the distal aperture of the sheath; and
securing the optical fiber and the beam-shaping insert to the sheath.

14. The method of claim 13, wherein securing the optical fiber and the beam-shaping insert to the sheath comprises using an adhesive.

15. The method of claim 13, wherein the beam-shaping insert comprises a reflective element, and wherein adjusting the orientation between the optical fiber and the beam-shaping insert comprises aligning the reflective element with an optical axis of the optical fiber, whereby an electromagnetic beam emitted from the optical fiber is reflected by the reflective element.

16. The method of claim 15, the sheath further comprising an aperture in the inner wall through which the electromagnetic beam is reflected.

17. The method of claim 13, wherein the optical fiber is in direct contact with the inner wall of the sheath.

18. The method of claim 13, wherein a torsional maintaining element is positioned about at least one of the optical fiber and the sheath near the proximal aperture of the sheath.

19. The method of claim 13, further comprising filling a central cavity defined by the inner wall of the sheath between the beam-shaping insert and the output face of the optical fiber with an optically transparent medium.

20. The method of claim 13, wherein the output face of the optical fiber is prepared at an angle of between about 0° and about 10° relative to an axis perpendicular to the optical axis of the optical probe.
